(19) United States
(12) Patent Application Publication
(10) Pub. No.: US 2011/0294131 A1
(43) Pub. Date: Dec. 1, 2011

(54) ANALYZER, AND CONTROL METHOD FOR ROTATION OF DISC

(76) Inventors: Kohshi Maeda, Tokai (JP); Takehiko Hosoiri, Hitachinaka (JP); Minoru Sano, Hitachinaka (JP); Masato Ishizawa, Hitachinaka (JP)

(21) Appl. No.: 13/147,106

(22) PCT Filed: Dec. 14, 2009
(86) PCT No.: PCT/JP2009/006828

§ 371 Date: Jul. 29, 2011

(30) Foreign Application Priority Data
Jan. 30, 2009 (JP) 2009-01896

(51) Int. Cl. C12Q 1/68; C12M 1/34

(52) U.S. Cl. 435/6.12; 435/287.2

(57) ABSTRACT

The present invention has an object to provide an automatic analyzer using a disc, which treats samples with different pretreatment times or samples with different measurement times requested at random with high throughput. The present invention relates to performing adjustment rotation, rotation returning to origin, and measurement rotation in specimen analysis using a disc having a plurality of sample positions on a circumference. The adjustment rotation is an operation for placing a desired sample position in a particular position for introducing a sample into the disc or discarding the sample. The rotation returning to origin is an operation for placing an origin of the disc in a particular position. The measurement rotation is an operation for rotating the disc at a predetermined speed for measuring a plurality of samples held by the disc.

### TECHNICAL FIELD

The present invention relates to an analyzer including a disc that can hold a plurality of samples.

### BACKGROUND ART

An analyzer including a disc that can hold a plurality of samples is known as an automatic analyzer for blood, urine, or the like. Such an analyzer generally includes a detector, a sample introduction mechanism, a reagent addition mechanism, a cleaning mechanism, or the like around the disc. Samples are continuously introduced into the disc from a particular position, and the detector is operated with rotation of the disc to detect sample signals with time.

As a driving method of a disc, the disc is generally rotated once or more in one direction within a predetermined time required for detecting sample signals with time. This allows the sample signals to be detected with time and allows samples to be continuously introduced even if a sample introduction position is fixed in a particular position.

As an example, for an analyzer that detects samples every 18 seconds using a disc on which 160 reaction cells are placed, the disc is rotated for 41 reaction cells every 4.5 seconds, and rotated for 165 reaction cells (reaction cells of the number for one rotation+five reaction cells) in 18 seconds. Thus, the reaction cells placed in a sample introduction position are changed for each measurement in order of No. 1, No. 42, No. 83, No. 124, No. 5, No. 46, No. 87, No. 128, . . . No. 199, No. 160 to achieve sample signal detection with time and continuous sample introduction.

JP Patent Publication (Kokai) No. 2003-21645A (Patent Literature 1) discloses an automatic analyzer that changes a rotational direction and an amount of movement of a disc.

### CITATION LIST

### Patent Literature

Patent Literature 1: JP Patent Publication (Kokai) No. 2003-21645A

### SUMMARY OF INVENTION

### Technical Problem

The inventors of the application have diligently studied nucleic acid analysis using a disc that can hold a plurality of samples, and obtained the following findings.

Before a sample containing nucleic acid is introduced into a disc, the sample needs to be subjected to pretreatment such as reaction solution adjustment, and a time required for the pretreatment may differ depending on analysis items requested. For example, a DNA amplification test and an RNA amplification test require different pretreatments. When a PCR method is used as a nucleic acid analysis method, DNA can be directly amplified, while for RNA, a process for synthesizing DNA from template RNA using reverse transcriptase is required, and pretreatment times are significantly different. When a Nucleic Acid Sequence based Amplification method (hereinafter referred to as an NASBA method) for detecting RNA as a template is used as a nucleic acid analysis method, RNA can be directly detected, while for DNA, a process for restriction enzyme treatment of template DNA is required, and pretreatment times are also significantly different. Also in a nucleic acid test including a nucleic acid extraction process, a pretreatment method differs depending on patient's specimens (blood, serum, plasma, sputum, urine, stool, or the like) or types of template nucleic acid to be extracted. Specifically, when the pretreatment method differs as described above, it is very difficult to continuously treat samples in the same time.

Thus, for an automatic analyzer that continuously analyzes samples using a disc, when assays with different pretreatment times are requested at random, samples cannot be introduced into the disc at constant timing. If the disc is controlled by a conventional driving method, samples are discretely introduced into the disc to reduce the maximum number of samples placed on the disc and reduce test throughput.

For analysis methods with the same pretreatment time, samples with different measurement times may be requested at random. For example, amplification efficiency of a gene region to be amplified differs depending on test items, and thus a time to reach a maximum value of amplification differs. More specifically, by the NASBA method, real time measurement is performed at a measurement interval of 30 seconds until amplification of nucleic acid reaches a maximum value. Measurement times are significantly different such that the measurement time for an HIV virus test (NucliSENS HIV-1 kit, bioMérieux SA) is 60 minutes and the measurement time for an Enterovirus test (NucliSENS Enterovirus kit, bioMérieux SA) is 180 minutes. Also in biochemical automatic analysis, a reaction time course differs depending on test items and thus a measurement time differs. Thus, for automatic analysis in which samples with a short measurement time and samples with a long measurement time are introduced into a disc at random, timing of completion of measurement differs for each sample. Therefore, in a conventional disc driving method, even if measurement is completed, a measured sample cannot be discarded until the measured sample stops at a sample discard position. Also, even if there is a different sample position into which a sample can be introduced, a next sample cannot be introduced until the discarded sample position reaches a sample introduction position.

Further, in an automatic analyzer using a disc, if analyses with different pretreatment times and analyses with different measurement times are requested at random, throughput is extremely reduced.

The present invention has an object to provide an automatic analyzer using a disc, which treats samples with different pretreatment times or samples with different measurement times requested at random with high throughput.

### Solution to Problem

The present invention relates to performing adjustment rotation, rotation returning to origin, and measurement rotation in specimen analysis using a disc having a plurality of sample positions on a circumference. The adjustment rotation is an operation for placing a desired sample position in a particular position for introducing a sample into the disc or discarding the sample. The rotation returning to origin is an operation for placing an origin of the disc in a particular position. The measurement rotation is an operation for rotating the disc at a predetermined speed for measuring a plurality of samples held by the disc.

### Advantageous Effects of Invention

According to the present invention, even if samples with different pretreatment times are requested at random, the samples can be introduced into the disc without space to increase throughput of random requests for analyses with different pretreatment times. Even if analyses with different measurement times are requested at random, samples can be quickly discarded after measurement of the samples is completed, allowing introduction of new samples and increasing throughput.

### BRIEF DESCRIPTION OF DRAWINGS

FIG. 1 is a schematic view illustrating a basic configuration of an automatic analyzer according to an embodiment.

FIG. 2 is a schematic view illustrating a reaction container conveying mechanism or the like in the embodiment.

FIG. 3 is a schematic view showing a relation between the automatic analyzer and peripheral devices in the embodiment.

FIG. 4 is a schematic view of a disc and peripheral mechanisms in the embodiment.

FIG. 5 is an operation flowchart of the disc, a detector, and a sample introduction/discard mechanism in the embodiment.

FIG. 6 is an operation flowchart from measurement start operation to measurement rotation in the embodiment.

FIG. 7 is an operation flowchart of a shield unit, a disc, a detector, and a sample introduction/discard mechanism in the embodiment.

### DESCRIPTION OF EMBODIMENTS

The inventors of the application have made various studies for a disc driving method that can continuously introduce or discard samples while maintaining maximum throughput in an automatic analyzer that is requested to perform analyses with different pretreatment times or different measurement times, and reached an analyzer and a method disclosed in an embodiment.

In the embodiment, a sample position on a disc into which a sample can be introduced is moved to an introduction position accessible to a mechanism that can introduce and discard a sample by adjustment rotation, and the sample is introduced by a sample introduction mechanism. Then, in order to maintain a fixed measurement interval for all samples introduced into the disc, the disc is moved to an origin as a starting point of measurement rotation by rotation returning to origin. Finally, the disc is rotated from the origin at a fixed speed to measure all the samples introduced. The adjustment rotation, rotation returning to origin, and measurement rotation are performed at a predetermined measurement interval determined by an analysis method.

In the embodiment, rotational speeds of the adjustment rotation and the rotation returning to origin are faster than a rotational speed of the measurement rotation. The adjustment rotation and the rotation returning to origin can be performed both to right and left so that the disc can be moved to a target position in a minimum travel distance. This ensures a long time required for introducing or discarding samples.

In an embodiment, an automatic analyzer is disclosed including: a disc having a plurality of sample positions that can hold samples on a circumference; a sample introduction device that introduces a sample into the disc; a detector that is placed around the disc and measures the sample placed on the disc; and a rotation control device that controls rotation of the disc, wherein the disc is rotated so as to move a sample position to an introduction position accessible to a sample introduction mechanism, then a sample is introduced into the sample position placed in the introduction position by the sample introduction mechanism, the disc is rotated so as to move an origin of the disc to a particular position, and then a plurality of samples on the disc are measured by the detector while the disc is rotated at a predetermined speed.

In an embodiment, an automatic analyzer is disclosed including: a disc having a plurality of sample positions that can hold samples on a circumference; a sample introduction device that introduces a sample into the disc and discards a sample from the disc; a detector that is placed around the disc and measures the sample placed on the disc; and a rotation control device that controls rotation of the disc, wherein the disc is rotated so as to move a sample position that holds a measured sample to an introduction/discard position accessible to a sample introduction/discard mechanism, then the sample is discarded from the sample position placed in the introduction/discard position by the sample introduction/discard mechanism, a sample is introduced into the sample position, the disc is rotated so as to move an origin of the disc to a particular position, and then the plurality of samples on the disc are measured by the detector while the disc is rotated at a predetermined speed.

In an embodiment, an automatic analyzer is disclosed including: a disc having a plurality of sample positions that can hold samples on a circumference; a sample introduction/discard device that introduces a sample into the disc and discards a sample from the disc; a detector that is placed around the disc and measures the sample placed on the disc; and a rotation control device that controls rotation of the disc, wherein the disc is rotated so as to move a sample position that holds a measured sample to a discard position accessible to a sample discard mechanism, then the sample is discarded from the sample position placed in the discard position by the sample discard mechanism, the disc is rotated so as to move the sample position to an introduction position accessible to a sample introduction mechanism, then a sample is introduced into the sample position placed in the introduction position by the sample introduction mechanism, the disc is rotated so as to move an origin of the disc to a particular position, and then a plurality of samples on the disc are measured by the detector while the disc is rotated at a predetermined speed.

In an embodiment, an automatic analyzer is disclosed including: a disc having a plurality of sample positions that can hold samples on a circumference; a sample discard device that can discard a sample from the disc; a detector that is placed around the disc and measures the sample placed on the disc; and a rotation control device that controls rotation of the disc, wherein the disc is rotated so as to move an origin of the disc to a particular position, then a plurality of samples on the disc are measured by the detector while the disc is rotated at a predetermined speed, the disc is rotated so as to move a sample position that holds a measured sample to a discard position accessible to a sample discard mechanism, and then the sample is discarded from the sample position placed in the discard position by the sample discard mechanism.

An embodiment discloses a control method for rotation of a disc, wherein in an analyzer including a disc having a plurality of sample positions that can hold samples on a circumference, a rotation control device that controls rotation of the disc rotates the disc so as to move a sample position to an introduction position accessible to a sample introduction mechanism, the sample introduction mechanism introduces a sample into the sample position placed in the introduction position, then the rotation control device rotates the disc so as to move an origin of the disc to a particular position, then the rotation control device rotates the disc at a predetermined speed, and a detector measures a plurality of samples on the disc.

An embodiment discloses a control method for rotation of a disc, wherein in an analyzer including a disc having a plurality of sample positions that can hold samples on a circumference, a rotation control device that controls rotation of the disc rotates the disc so as to move a sample position that holds a measured sample to an introduction/discard position accessible to a sample introduction/discard mechanism, the sample introduction/discard mechanism discards the sample from the sample position placed in the introduction/discard position and introduces a sample into the sample position, then the rotation control device rotates the disc so as to move an origin of the disc to a particular position, and then the rotation control device rotates the disc at a predetermined speed, and a detector measures a plurality of samples on the disc.

An embodiment discloses a control method for rotation of a disc, wherein in an analyzer including a disc having a plurality of sample positions that can hold samples on a circumference, a rotation control device that controls rotation of the disc rotates the disc so as to move a sample position that holds a measured sample to a discard position accessible to a sample discard mechanism, the sample discard mechanism discards the sample from the sample position placed in the discard position, then the rotation control device rotates the disc so as to move the sample position to an introduction position accessible to a sample introduction mechanism, the sample introduction mechanism introduces a sample into the sample position placed in the introduction position, then the rotation control device rotates the disc so as to move an origin of the disc to a particular position, then the rotation control device rotates the disc at a predetermined speed, and a detector measures a plurality of samples on the disc.

An embodiment discloses a control method for rotation of a disc, wherein in an analyzer including a disc having a plurality of sample positions that can hold samples on a circumference, a rotation control device that controls rotation of the disc rotates the disc so as to move an origin of the disc to a particular position, then a detector measures a plurality of samples on the disc while the disc is rotated at a predetermined speed, the rotation control device rotates the disc so as to move a sample position that holds a measured sample to a discard position accessible to a sample discard mechanism, and the sample discard mechanism discards the sample from the sample position placed in the discard position.

The embodiment discloses that a rotational speed and/or a rotational direction of the disc are variable.

The embodiment discloses that the rotational direction is controlled so as to minimize a travel time when the origin of the disc is moved to a particular position.

The embodiment discloses that when a particular sample position of the disc is moved to the introduction position, the rotational direction is controlled to minimize the travel time.

The embodiment discloses that when the particular sample position of the disc is moved to the discard position, the rotational direction is controlled to minimize the travel time.

The embodiment discloses that the sample held by the disc is a pretreated specimen.

The embodiment discloses that the sample held by the disc is a reaction solution that can amplify target nucleic acid at a desired temperature.

The embodiment discloses that the sample introduction mechanism or the sample introduction/discard mechanism includes a reaction container conveying mechanism that conveys a reaction container holding a sample to the disc.

The embodiment discloses that the sample discard mechanism or the sample introduction/discard mechanism includes a reaction container conveying mechanism that conveys a reaction container holding a sample from the disc.

Now, the foregoing and other novel features and advantages will be described with reference to the drawings. The drawings are used for understanding the invention, and do not limit the scope of right. The embodiments may be combined.

### Embodiments

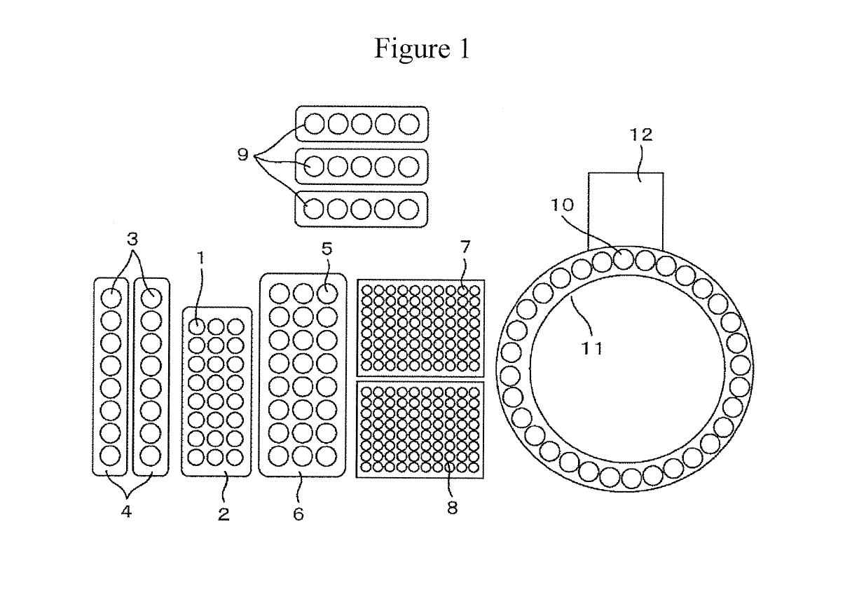

FIG. 1 shows a basic configuration of an automatic analyzer according to an embodiment. The analyzer includes a specimen container rack 2 in which a specimen container 1 containing a specimen to be tested can be set, a reagent rack 4 housing a plurality of reagent containers 3 containing reagents that react with various ingredients of the specimen, a reaction container rack 6 housing a reaction container 5 in which the specimen is mixed with the reagent, a dispensing chip rack 8 housing a dispensing chip 7, a plurality of incubators 9 set to two or more different temperatures, a disc 11 on which a plurality of reaction container carriers 10 are placed on a circumference, and a detector 12 that detects a reaction.

The reaction container 5 in this embodiment may be any container that has little influence on detection performance, and preferably a 0.2 ml single tube (Greiner Bio-One Co. Ltd., Germany).

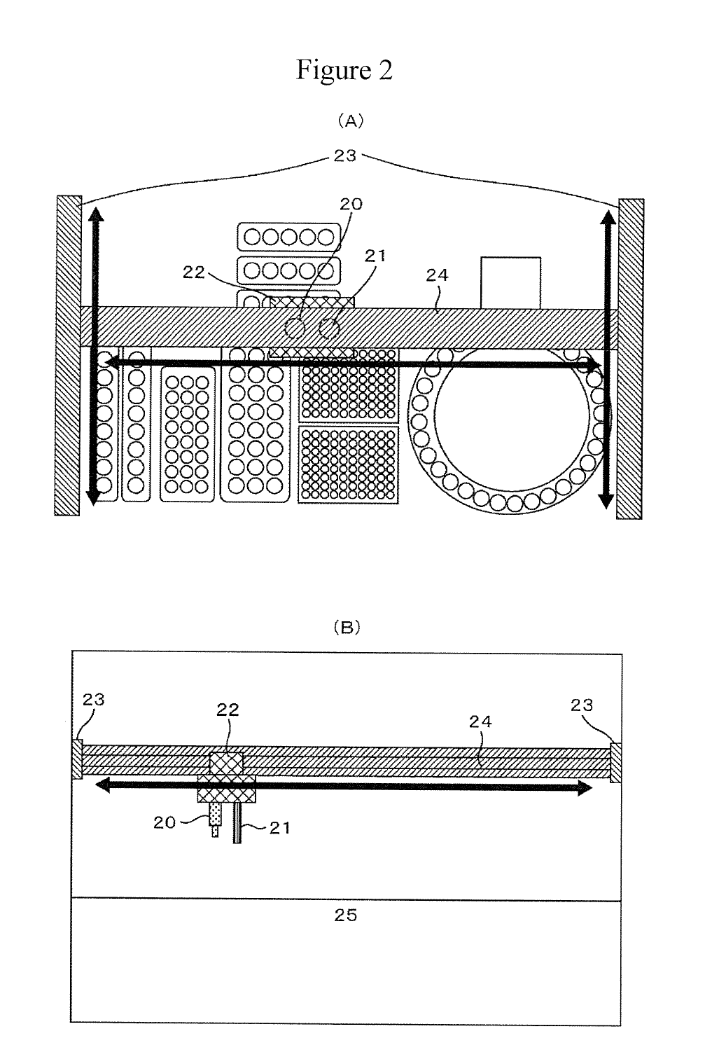

FIG. 2 is a schematic view illustrating a dispensing probe 20 for dispensing a specimen or a reagent between racks, a reaction container conveying mechanism 21 for conveying a reaction container, a head 22 that connects the dispensing probe 20 and the reaction container conveying mechanism 21, and a side rail 23 and a center rail 24 for moving the head in X-axis and Y-axis directions.

Now, a basic operation of the analyzer for automatic analysis of a specimen will be described. The dispensing probe 20 is moved to a position of the dispensing chip rack 8 by operations of the side rail 23 and the center rail 24, and the dispensing chip 7 is mounted to the dispensing probe 20. The dispensing probe 20 to which the dispensing chip 7 is mounted takes out a specimen from the specimen container 1, and ejects the specimen into the reaction container 5. Then, the dispensing chip 7 is changed, a reagent is taken out from the reagent container 3 and ejected to the reaction container 5, and the dispensing probe 20 repeats suction and ejection to mix a reaction solution. The reaction container 5 containing the reaction solution is conveyed to one of the incubators 9 by the reaction container conveying mechanism 21 to change a temperature of the reaction solution. After the reaction container 5 is held for a predetermined time in the incubator, the reaction container 5 is conveyed to another incubator 9 by the reaction container conveying mechanism to change the temperature of the reaction solution, and is similarly held for a predetermined time in the incubator. The conveyance between the incubators is performed under a condition determined by an analysis method. According to the determined analysis method, after or during a temperature change between the incubators, the dispensing probe 20 to which the dispensing chip 7 is mounted may take out a reagent from the reagent container 3 and eject the reagent to the reaction container 5. After the temperature change, the reaction container conveying mechanism 21 conveys the reaction container 5 containing a pretreated specimen to a standby region near a sample introduction/discard mechanism. The pretreated specimen includes a specimen to be tested and a reaction solution in which a reagent required for a reaction is mixed. For example, for nucleic acid analysis, the pretreated specimen is a reaction solution that can amplify target nucleic acid as a specimen to be tested at a desired temperature.

The sample introduction/discard mechanism places the reaction container 5 in the standby region in the predetermined reaction container carrier 10 placed on the disc 11. Also, as required, the reaction container 5 placed on the predetermined reaction container carrier 10 is discarded. The reaction container carrier 10 is maintained at a temperature at which the reaction is most effectively developed, by a temperature control mechanism of the disc. The reaction is detected by the detector 12. In order to prevent degradation of samples by light or entry of ambient light in detection, the disc 11 and the detector 12 are entirely shielded from ambient light.

In this embodiment, as a conveying mechanism that conveys a reaction container, a conveying mechanism that conveys both the dispensing probe and the reaction container is disclosed, but the dispensing probe and the reaction container may be handled by different conveying mechanisms. In this embodiment, only one conveying mechanism is provided, but a plurality of conveying mechanisms may be placed correspondingly to a plurality of constant temperature mechanisms or analysis mechanisms. In this embodiment, the conveying mechanism and the sample introduction/discard mechanism are separate, but may be integrated so that the conveying mechanism directly conveys the reaction container 5 to the reaction carrier 10 on the disc 11.

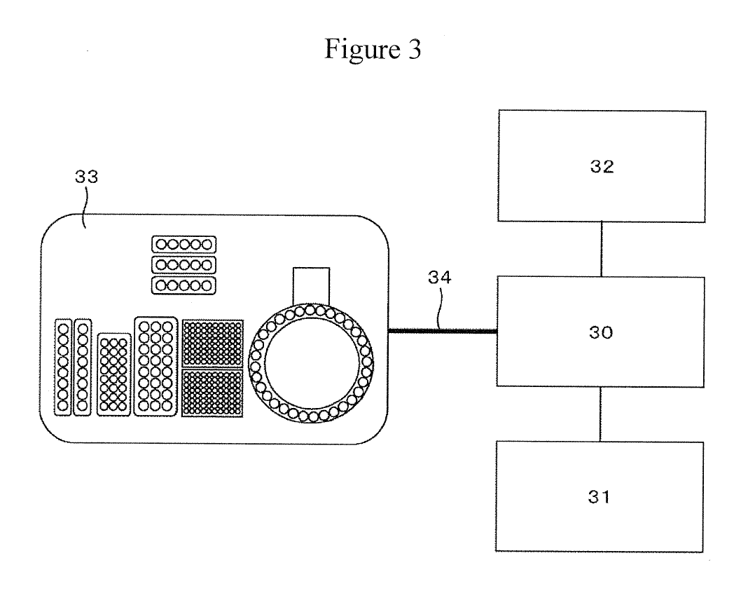

FIG. 3 shows a relation between the automatic analyzer and peripheral devices. To the analyzer, a storage device 30 that stores detection data, a calculator 31 that calculates the stored detection data, and a monitor 32 that outputs a calculation result. The peripheral devices may be connected to the automatic analyzer by a communication cable 34 and independently function, or may be included in the automatic analyzer. The detection data detected by the detector 12 is transmitted to the storage device 30 and calculated by the calculator 31, and the detection result is output to the monitor 32.

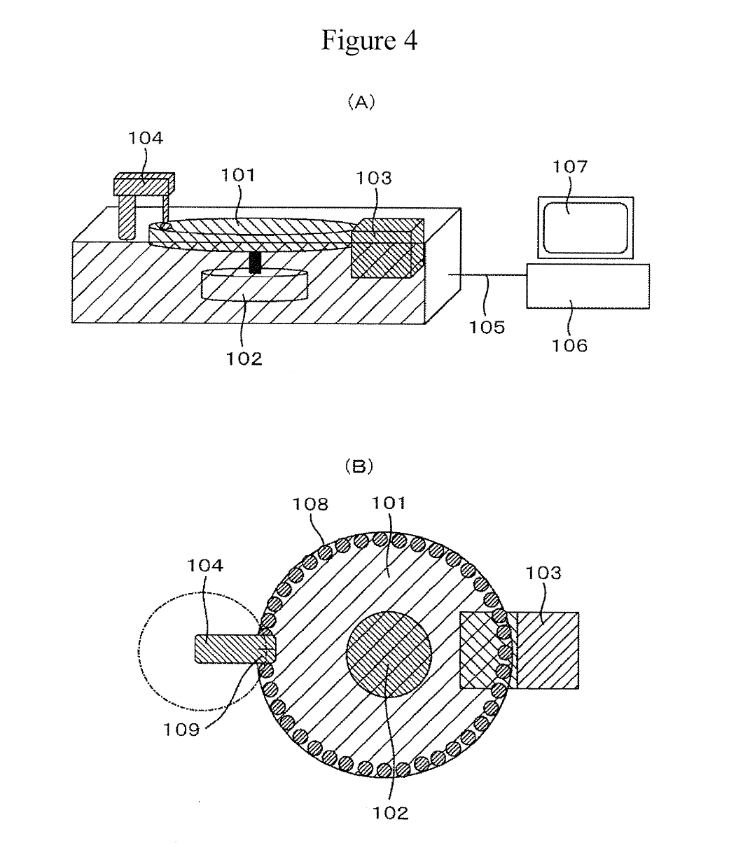

FIG. 4 schematically shows peripheral mechanisms of the disc. The analyzer includes a disc 101, a drive unit 102 that rotates the disc 101, a detector 103 for analyzing a pretreated specimen in the reaction container, a mechanism that introduces a sample into the disc, and a sample introduction/discard mechanism 104 that conveys the reaction container to a predetermined reaction container carrier 10 on the disc 11, and discards a reaction container held by the predetermined reaction container carrier 10. The mechanism is controlled by a control PC 106 including the storage device 30 and the calculator 31 via a communication cable 105.

The disc in this embodiment is a rotating disc as shown in FIG. 4(B), and includes a plurality of reaction carriers at an edge where the reaction containers can be held. A position of each reaction carrier on the disc can be regarded as a sample position. A plurality of sample containers may be provided at the edge of the disc, and a pretreated specimen may be directly dispensed to the sample container. In this case, a position with the sample container can be regarded as a sample position. In the disc 101, an origin (on a disc side) as a starting point of measurement rotation is virtually provided. The origin may be physically provided by marking a spot or the like on the disc 101.

The sample introduction/discard mechanism in this embodiment introduces a reaction container 5 holding a pretreated specimen into a particular reaction carrier on the disc, and lifts and discards a reaction container 5 held by a particular reaction carrier. In other words, the sample introduction/discard mechanism is a reaction container conveying mechanism, and conveys a reaction container to a reaction carrier placed on a sample introduction/discard position 109 set in a particular position on an analyzer body, and conveys a reaction container from a reaction carrier.

The sample introduction/discard mechanism is not limited to a mechanism that conveys a reaction container. For example, when a reaction container is directly provided on the disc, the sample introduction/discard mechanism may directly dispense a pretreated specimen to the reaction container and suck an unnecessary specimen from the container. In this case, a cleaning mechanism of the reaction container is desirably provided in the sample introduction/discard mechanism. Specifically, “sample introduction” herein refers to placement of a reaction container using the reaction container conveying mechanism, and also refers to addition of a specimen to the disc by sample dispensing in some cases. Also, “sample discard” herein refers to removal of a reaction container from the disc using the reaction container conveying mechanism, and also refers to suction of a specimen from the disc, or cleaning of the disc after suction in some cases.

The sample introduction mechanism and the sample discard mechanism may be separately provided. This may increase throughput. In this case, it may be allowed that the sample introduction/discard position is divided, a position where the sample introduction mechanism conveys a reaction container to a reaction carrier is a sample introduction position, and a position where the sample discard mechanism conveys a reaction container from a reaction carrier is a sample discard position. Further, even when an integral sample introduction/discard mechanism is used, the sample introduction position and the sample discard position may be separate.

The drive unit 102 in this embodiment controls a rotational angle of the disc 101, and rotates the disc 101 at a fixed speed. For a rotational direction, a clockwise direction and a counterclockwise direction may be selected as required, but is basically the clockwise direction. Depending on the intended use, the rotational direction may be limited to one direction. The rotational speed is variable depending on the intended use, but may be substantially constant depending on the intended use.

The drive unit 102 stops the disc 101 in desired states as required. One of the desired states is an origin state, and the other is a sample introduction/discard state.

The origin state is a state where a rotational angle of the disc 101 is in an initial state. In other words, the origin (on the disc side) of the disc 101 is a particular position with respect to the analyzer body. The origin state is used in “rotation returning to origin” described later or the like.

The sample introduction/discard state is a state where a desired reaction carrier is located in the sample introduction/discard position. In other words, a desired sample position is located in the sample introduction/discard position. The sample introduction/discard state is used in “sample introduction”, “sample discard”, or the like described later. When the sample introduction position and the sample discard position are separate, the state is separated into a sample introduction state and a sample discard state.

A detector 103 in this embodiment analyzes a sample in a reaction container held by the reaction carrier on the disc 101, and is provided in an upper, lower, or side portion of the disc 101. Any detector may be used that can analyze a sample to be measured. For example, when a sample labeled with fluorescent dye is analyzed, a detector is provided that detects an excitation light source and fluorescent emission. When an absorbance change is to be measured, a light source lamp and a photometer are provided.

The sample in this embodiment refers to an object to be measured detected by the detector, and may be any object to be measured that can be directly or indirectly detected by the detector. Specifically, when the object to be measured is a biological specimen, and a specimen ingredient is analyzed by an enzyme reaction, the sample includes a biological specimen ingredient and a reaction solution of a mixture of an enzyme liquid and a substrate liquid required for an enzyme reaction. More specifically, when nucleic acid as a biological specimen ingredient is amplified and detected, the sample includes nucleic acid extracted from the biological specimen ingredient by a pretreatment process and an amplification reaction solution such as primer or enzyme for amplifying the nucleic acid. There are a plurality of amplification methods of nucleic acid such as a PCR method, a LAMP method, a TRC method, a NASBA method, or a TMA method, but in the present invention, any method may be used that allows detection using the detector 103, not limited to a detailed measurement example described below.

The control PC 106 can control the drive unit 102 (disc 101), the detector 103, and the sample introduction/discard mechanism. The control PC 106 preferably includes a measurement data storage function, a calculation function, and a measurement result display function. The control PC 106 preferably includes a communication function, and conveys a calculation result to a different device, for example, a device having a printing function or a device that manages test data.

A driving method of the disc will be described. Rotation patterns of the disc in the analyzer can be broadly classified into measurement rotation, adjustment rotation, and rotation returning to origin.

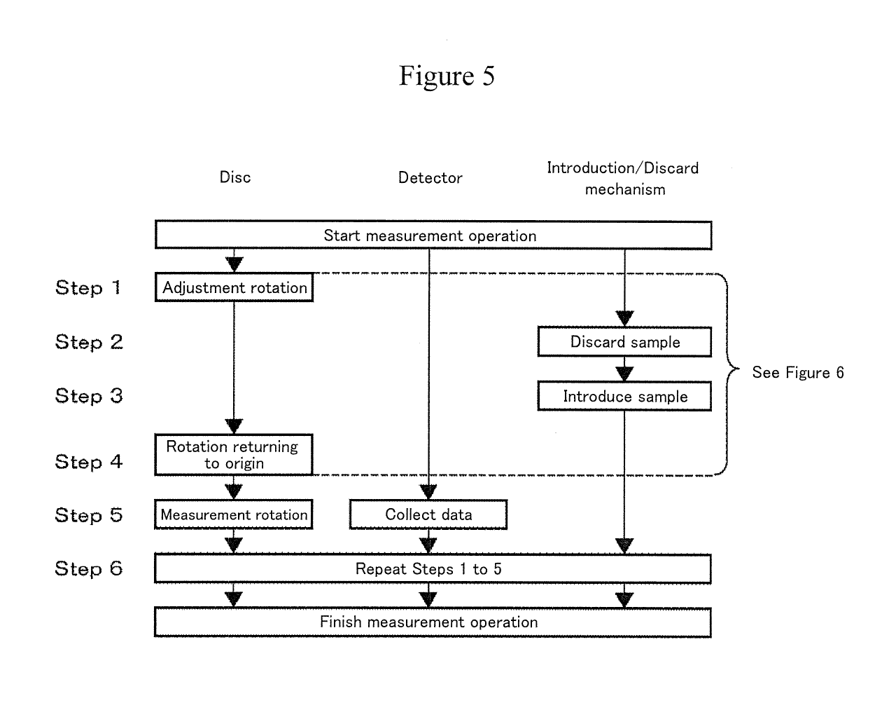

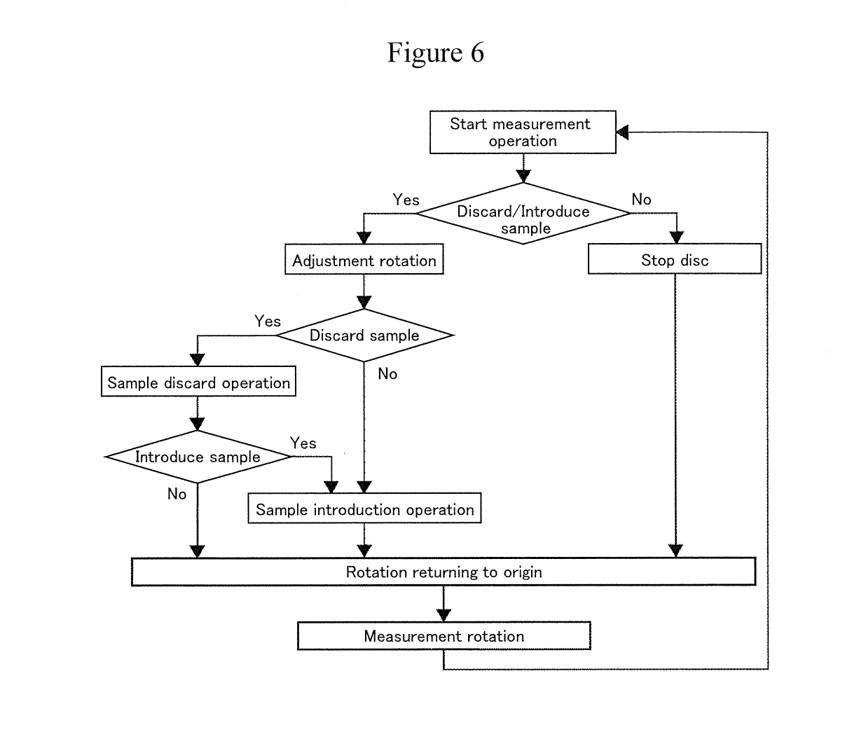

FIG. 5 shows a summary of an operation flow of the disc, the detector, and the sample introduction/discard mechanism. FIG. 6 shows a detailed operation flow from measurement start operation to measurement rotation.

In the analyzer, adjustment rotation is performed as driving operation of the disc after measurement operation is started. If there is a specimen to be discarded or a pretreated specimen to be introduced when the measurement operation is started, the specimen is discarded or introduced, and the rotation returning to origin is performed. If there is no specimen to be discarded or pretreated specimen to be introduced when the measurement operation is started, the disc is stopped for a time assigned to adjustment rotation or sample introduction/discard. Then, measurement rotation is performed to measure a sample, and data of a pretreated specimen is collected. This operation is repeated to analyze many samples.

In FIG. 5, Steps 1 to 5 are included in one measurement cycle. Measurement in the measurement cycle is repeatedly performed. The number of repetitions depends on measurement time.

The “adjustment rotation” in this embodiment refers to rotation for moving a sample position where a sample can be introduced to a sample introduction position. The sample position where a sample can be introduced refers to a sample position where there is no specimen or a sample position where an introduced specimen has been measured. When there is the measured specimen, the sample introduction/discard mechanism discards the measured sample in the disc, and then introduces a new pretreated specimen.

The adjustment rotation is performed, and thus new pretreated specimens can be introduced into any sample positions on the disc without space if analysis of a pretreated specimen is requested at any timing. In whatever order pretreated specimens with different measurement times are introduced into the disc, a pretreated specimen measured earliest can be successively discarded.

The drive unit selects a rotational direction and a rotational speed of the disc so that a desired sample position of the disc can be rotated to the sample introduction/discard position in a minimum distance. The rotational speed variable function and minimization of the rotational distance allow quick adjustment rotation and ensure a longer time for sample introduction and/or sample discard.

The “rotation returning to origin” in this embodiment is an operation for returning the position of the disc to the origin after measurement rotation or adjustment rotation in order to start rotation from the origin in next measurement rotation. By the rotation returning to origin, if the disc is stopped in any position by measurement rotation or adjustment rotation, the disc is returned to the origin before measurement rotation, and thus a pretreated specimen in any position in the disc can be reliably measured at a certain interval.

The drive unit selects the rotational direction and the rotational speed of the disc so that the disc quickly enters an origin state. The rotational speed variable function and minimization of the rotational distance allow quick rotation returning to origin and ensure a longer time for sample introduction and/or sample discard. When the disc after the adjustment rotation and the measurement rotation enters the origin state, the rotation returning to origin is not performed, and the disc is stopped during a time assigned to the rotation returning to origin.

The “measurement rotation” in this embodiment is rotation for measuring a pretreated specimen introduced into the sample position, and an operation for starting rotation with the origin as a starting point, and stopping the rotation after all pretreated specimens introduced are measured. The measurement rotation is always started from the origin state, the disc is rotated at the same speed, and the pretreated specimen is measured at the same timing, and thus a pretreated specimen in any sample position in the disc can be reliably measured at a regular interval.

A time cycle for performing the adjustment rotation, the sample introduction and/or discard, the rotation returning to origin, and the measurement rotation is determined from a measurement interval required by an analysis method. The measurement interval in this embodiment is a time between detection points when the pretreated specimen is detected in real time. More specifically, a measurement interval of the NASBA method is 30 seconds, and a measurement interval of biochemical analysis is 18 seconds. Of course, any measurement interval may be set as long as the analysis method accepts. When the measurement interval is X, a time for adjustment rotation is A, a time for sample introduction and/or discard is B, a time for rotation returning to origin is C, and a time of measurement rotation is D, a relationship between the operation times is expressed by Expression 1.

A+B+C+D=X (1)

Specifically, if the rotational speed and the rotational direction are controlled in the adjustment rotation or the rotation returning to origin, and the time A of the adjustment rotation or the time C of the rotation returning to origin is shortened, a longer time B required for introducing or discarding a sample can be ensured.

As described above, according to this embodiment, even if protocols with different pretreatment times are applied to a specimen at random, the reaction containers can be placed on the disc without space. Even if test items with different measurement times are requested at random, a measured sample can be quickly discarded. Further, the adjustment rotation and the rotation returning to origin can be performed in a short time, thereby increasing a time assigned to discarding or placing the reaction container.

As a more detailed example, a disc driving method when the analyzer is used for nucleic acid analysis by a NASBA method will be described below.

### 1. Measurement Condition

Table 1 shows a measurement condition of the NASBA method as one of nucleic acid analysis methods, Table 2 shows reagents and specimens used, and Table 3 shows a specimen adjustment method.

TABLE 1
Measurement condition of NASBA method

| Item | Contents |
|---|---|
| Test protocol | DNA protocol, RNA protocol |
| Detection reagent | fluorescent dye (FAM, ROX) |
| Measurement interval (second) | 30 |
| Measurement time (minute) | 30, 60, 90, 120, 150, 180 |

TABLE 2
Reagents and speciments of NASBA method

| Item | Contents |
|---|---|
| Reagent used | NucliSENS HIV-1 kit(RNA), NucliSENS HSV-1 kit |
| | (DNA) |
| Specimens to | HIV-1 kit: positive control RNA nucleic acid, internal |
| be tested | standard RNA nucleic acid, |
| | HSV-1 kit: positive control DNA nucleic acid, internal |
| | standard DNA nucleic acid |

TABLE 3
Sample preparation method using reagents in Table 2

| | | | Device |
|---|---|---|---|
| Process | HIV-1 kit | HSV-1 kit | used |
| Reagent | 90 ul of diluted solution is | 90 ul of diluted solution is | |
| preparation | added to a lyophilized | added to a lyophilized | |
| | reagent and stirred and | reagent and stirred and | |
| | dissolved by a dispensing chip. | dissolved by a dispensing | |
| | | chip. After dissolved, 85 ul | |
| | | of the solution is sucked and | |
| | | added to lyophilized control | |
| | | enzyme and stirred and dissolved. | |
| Enzyme | 45 ul of diluted solution is added to lyophilized enzyme and | | |
| prepartion | stirred and dissolved by a dispensing chip. | | |
| Reaction solution | 10 ul of reagent is dispensed into a reaction container, then 5 | | 0.2 ml |
| preparation | ul of specimen is added, and they are stirred by a dispansing chip. | | minute tube |
| Control enzyme | No | 37° C. 15 minutes | EasyQ |
| treatment | | | Incubator |
| Heating | 65° C. 2 minutes => 41° C. | 95° C. 5 minutes => 41° C. | |
| denaturation | | | |
| treatment | | | |

| | |
|---|---|
| Enzyme addition | After 41° C. is reached, 5 ul of enzyme is added and stirred |
| | with a cap being closed. |

### 2. Configuration of Analyzer

Table 4 shows details of a configuration of the analyzer when nucleic acid analysis is performed by a NASBA method.

TABLE 4
Configuration of analyzer when NASBA method is performed

| Item | Contents |
|---|---|
| Disc | A disc configured so that a minute reaction container of 0.2 ml can |
| | be placed thereon, and a bottom and a side surface of the reaction |
| | container are open |
| Disc drive mechanism | A mechanism that can drive the disc by rotation at a constant |
| | speed, rotation in opposite directions, and rotation at variable |
| | speed |
| Disc heating mechanism | Control to NASBA reaction temperature (41° C.) |
| Dectector | Detector that applies an excitation light to a bottom of the reaction |
| | container, excites fluorecent dye, and detects from a side surface |
| | using a filter optimum for each fluorecent wavelength. There are |
| | two types of detectors for FAM and ROX, which are arranged on a |
| | side surface of the disc. |
| Sample | The mechanism has a function of holding, lifting and lowering the |
| introduction/discard | reaction container. The mechanism can continuously perform |
| mechanism | introduction and discard. |
| Ambient light shield unit | The unit is configured to entirely cover the disc, the drive |
| | mechanism, the detector, and the disc heating mechaism. The |
| | unit includes an automatic opening/closing shutter in a sample |
| | introduction portion. |
| Control PC | The control PC performs control of the disc drive mechanism, |
| | temperature control, shutter control, control of the sample |
| | introduction/discard mechanism, obtaining data from the detector, |
| | and a date analysis function. |

### 3. Detection Operation

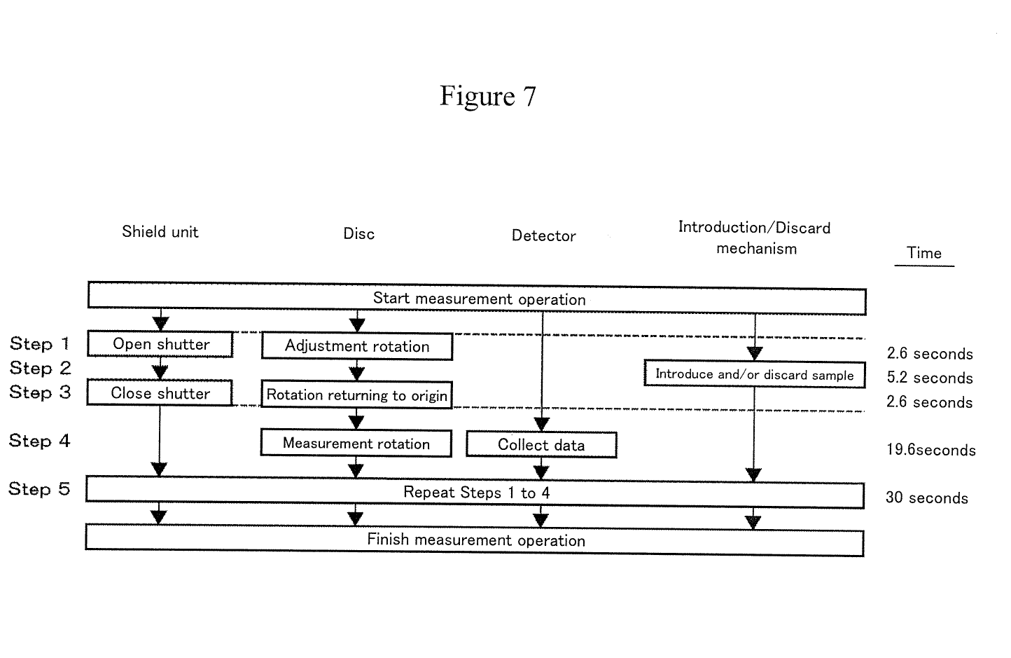

An operation of the analyzer when nucleic acid is analyzed by the NASBA method will be described. FIG. 7 shows a detailed operation flow of a shield unit, the disc, the detector, and the sample introduction/discard mechanism. In this example, an automatic opening/closing shutter provided in a sample introduction portion is opened at the time of adjustment rotation, and closed at the time of rotation returning to origin. This can prevent contamination of a specimen. For time assigned to each operation, the adjustment rotation is 2.6 seconds, the sample introduction and/or discard is 5.2 seconds, the rotation returning to origin is 2.3 seconds, and the measurement rotation is 19.6 seconds. Specifically, a pretreated specimen is introduced or discarded and measured in units of 30 seconds.

### INDUSTRIAL APPLICABILITY

The present invention can be applied to a nucleic acid analyzer that measures pretreated specimens with different pretreatment times or measurement times at random. Nucleic acid analysis may be applied to a constant temperature amplification method for amplifying nucleic acid at a constant temperature, and also a nucleic acid amplification method for denaturing or annealing by changing a temperature such as a PCR method. Further, besides the nucleic acid analysis, the present invention may be applied to an automatic analyzer of a biological specimen such as blood or urine using an antigen-antibody reaction.

### REFERENCE SIGNS LIST

1, 3 reagent container
2 specimen container rack
4 reagent rack
5 reaction container
6 reaction container rack
7 dispensing chip
8 dispensing chip rack
9 incubator
10 reaction container carrier
11, 101 disc
12, 103 detector
20 dispensing probe
21 reaction container conveying mechanism
22 head
23 side rail
24 center rail
25 analysis table surface
30 storage device
31 calculator
32, 107 monitor
33 automatic analyzer
34, 105 communication cable
102 drive unit
104 sample introduction/discard mechanism
106 control PC
108 sample position
109 sample introduction/discard position

1. An automatic analyzer comprising:
a disc having a plurality of sample positions that can hold samples on a circumference;
a sample introduction device that introduces a sample into the disc;
a detector that is placed around the disc and measures the sample placed on the disc; and
a rotation control device that controls rotation of the disc,
wherein the disc is rotated so as to move a sample position to an introduction position accessible to a sample introduction mechanism, then a sample is introduced into the sample position placed in the introduction position by the sample introduction mechanism,
the disc is rotated so as to move an origin of the disc to a particular position, and then a plurality of samples on the disc are measured by the detector while the disc is rotated at a predetermined speed.

2. An automatic analyzer comprising:
a disc having a plurality of sample positions that can hold samples on a circumference;
a sample introduction device that introduces a sample into the disc and discards a sample from the disc;
a detector that is placed around the disc and measures the sample placed on the disc; and
a rotation control device that controls rotation of the disc,
wherein the disc is rotated so as to move a sample position that holds an measured sample to an introduction/discard position accessible to a sample introduction/discard mechanism, then the sample is discarded from the sample position placed in the introduction/discard position by the sample introduction/discard mechanism, a sample is introduced into the sample position,
the disc is rotated so as to move an origin of the disc to a particular position, and then the plurality of samples on the disc are measured by the detector while the disc is rotated at a predetermined speed.

3. An automatic analyzer comprising:
a disc having a plurality of sample positions that can hold samples on a circumference;
a sample introduction/discard device that introduces a sample into the disc and discards a sample from the disc;
a detector that is placed around the disc and measures the sample placed on the disc; and
a rotation control device that controls rotation of the disc,
wherein the disc is rotated so as to move a sample position that holds an measured sample to a discard position accessible to a sample discard mechanism, then the sample is discarded from the sample position placed in the discard position by the sample discard mechanism,
the disc is rotated so as to move the sample position to an introduction position accessible to a sample introduction mechanism, then a sample is introduced into the sample position placed in the introduction position by the sample introduction mechanism,
the disc is rotated so as to move an origin of the disc to a particular position, and then a plurality of samples on the disc are measured by the detector while the disc is rotated at a predetermined speed.

4. An automatic analyzer comprising:
a disc having a plurality of sample positions that can hold samples on a circumference;
a sample discard device that can discard a sample from the disc;
a detector that is placed around the disc and measures the sample placed on the disc; and
a rotation control device that controls rotation of the disc,
wherein the disc is rotated so as to move an origin of the disc to a particular position, then a plurality of samples on the disc are measured by the detector while the disc is rotated at a predetermined speed,
the disc is rotated so as to move a sample position that holds a measured sample to a discard position accessible to a sample discard mechanism, then the sample is discarded from the sample position placed in the discard position by the sample discard mechanism.

5. The automatic analyzer according to any one of claims 1 to 4, wherein a rotational speed and/or a rotational direction of the disc are variable.

6. The automatic analyzer according to any one of claims 1 to 4, wherein the rotational direction is controlled so as to minimize a travel time when the origin of the disc is moved to a particular position.

7. The automatic analyzer according to any one of claims 1 to 3, wherein when a particular sample position of the disc is moved to the introduction position, the rotational direction is controlled to minimize the travel time.

8. The automatic analyzer according to any one of claims 2 to 4, wherein when the particular sample position of the disc is moved to the discard position, the rotational direction is controlled to minimize the travel time.

9. The automatic analyzer according to any one of claims 1 to 4, wherein the sample is a pretreated specimen.

10. The automatic analyzer according to any one of claims 1 to 4, wherein the sample is a reaction solution that can amplify target nucleic acid at a desired temperature.

11. The automatic analyzer according to any one of claims 1 to 3, wherein the sample introduction mechanism or the sample introduction/discard mechanism includes a reaction container conveying mechanism that conveys a reaction container holding a sample to the disc.

12. The automatic analyzer according to any one of claims 2 to 4, wherein the sample discard mechanism or the sample introduction/discard mechanism includes a reaction container conveying mechanism that conveys a reaction container holding a sample from the disc.

13. A control method for rotation of a disc having a plurality of sample positions that can hold samples on a circumference,
wherein a rotation control device that controls rotation of the disc rotates the disc so as to move a sample position to an introduction position accessible to a sample introduction mechanism,
the sample introduction mechanism introduces a sample into the sample position placed in the introduction position, then the rotation control device rotates the disc so as to move an origin of the disc to a particular position, and then
the rotation control device rotates the disc at a predetermined speed, and a detector measures a plurality of samples on the disc.

14. A control method for rotation of a disc having a plurality of sample positions that can hold samples on a circumference,
wherein a rotation control device that controls rotation of the disc rotates the disc so as to move a sample position that holds a measured sample to an introduction/discard position accessible to a sample introduction/discard mechanism,
the sample introduction/discard mechanism discards the sample from the sample position placed in the introduction/discard position and introduces a sample into the sample position, then the rotation control device rotates the disc so as to move an origin of the disc to a particular position, and then
the rotation control device rotates the disc at a predetermined speed, and a detector measures a plurality of samples on the disc.

15. A control method for rotation of a disc having a plurality of sample positions that can hold samples on a circumference,
wherein a rotation control device that controls rotation of the disc rotates the disc so as to move a sample position that holds a measured sample to a discard position accessible to a sample discard mechanism,
the sample discard mechanism discards the sample from the sample position placed in the discard position, then the rotation control device rotates the disc so as to move the sample position to an introduction position accessible to a sample introduction mechanism,
the sample introduction mechanism introduces a sample into the sample position placed in the introduction position, then the rotation control device rotates the disc so as to move an origin of the disc to a particular position,
then the rotation control device rotates the disc at a predetermined speed, and a detector measures a plurality of samples on the disc.

16. A control method for rotation of a disc having a plurality of sample positions that can hold samples on a circumference,
wherein a rotation control device that controls rotation of the disc rotates the disc so as to move an origin of the disc to a particular position, then a detector measures a plurality of samples on the disc while the disc is rotated at a predetermined speed,
the rotation control device rotates the disc so as to move a sample position that holds a measured sample to a discard position accessible to a sample discard mechanism, and the sample discard mechanism discards the sample from the sample position placed in the discard position.
